(19) United States
(12) Patent Application Publication
(10) Pub. No.: US 2004/0220259 A1
(43) Pub. Date: Nov. 4, 2004

(54) Topical treatment of dermatological disorders associated with reactive or dilated blood vessels

(76) Inventors: Ruey J. Yu, Chalfont, PA (US); Eugene J. Van Scott, Abington, PA (US)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(21) Appl. No.: 10/817,479

(22) Filed: Apr. 2, 2004

Related U.S. Application Data
(60) Provisional application No. 60/460,322, filed on Apr. 4, 2003.

(51) Int. Cl. A61K 31/366; A61K 31/19

(52) U.S. Cl. 514/460; 514/557

(57) ABSTRACT

The invention provides a method of topically treating a dermatological disorder. The method includes topically applying a therapeutically effective amount of a cosmetic or dermatological composition to an affected area of the skin. The composition includes at least one compound that is (i) a polyhydroxy-aldonic acid, (ii) a polyhydroxy-aldonic lactone, (iii) a polyhydroxy-alduronic acid, (iv) a polyhydroxy-alduronic lactone, (v) a polyhydroxy-aldaric acid; (vi) a polyhydroxy-aldaric lactone, and (vii) an organic acid lactone having two or more hydroxyl or ketohydroxyl groups. The dermatological disorder treated is one associated with reactive or dilated blood vessels.

Also included in the invention are methods of treating dermatological disorders associated with reactive blood vessels that include topical application of a therapeutically effective amount of a composition. The composition includes at least one compound selected from a compound represented by formula (I):

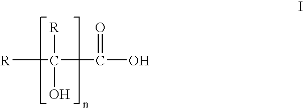

by formula (II):

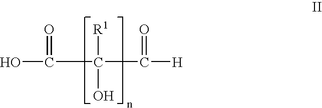

and by formula (III)

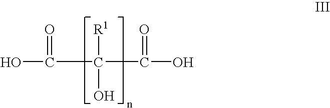

and a lactone derived from an organic acid having two or more hydroxyl or ketohydroxyl groups.

### CROSS-REFERENCE TO RELATED APPLICATIONS

This application claims the benefit of U.S. patent application Ser. No. 60/460,322 filed on Apr. 4, 2003.

### BACKGROUND OF THE INVENTION

Various dermatological disorders afflict most of the population at one time or another in the course of an individual's lifetime. Most of these disorders are not life threatening, but often involve prolonged or chronic pain, discomfort, and can cause social embarrassment because of the unsightly appearance of affected skin. Often, as a result, the afflicted individual may suffer social isolation or depression, in addition to the physiological symptoms of the disorder.

Because the etiologies of many common dermatological disorders are not known or well elucidated, there is often no cure to offer the patient. However, many disorders, although perhaps not mechanistically related, share one or more common symptoms, e.g., inflammation, dilation, constrictions or other disturbances of the blood vessels and capillaries of the skin. These symptoms can be treated to alleviate discomfort and/or aesthetically improve unsightly skin areas.

Thus, there is a need in the art for methods and compositions that can be used to treat dermatological disorders associated with the inflammation, dilation, constrictions, or other disturbances of the blood vessels and capillaries of the skin.

### BRIEF SUMMARY OF THE INVENTION

The invention provides a method of topically treating a dermatological disorder. The method includes topically applying a therapeutically effective amount of a cosmetic or dermatological composition to an affected area of the skin. The composition includes at least one compound that is (i) a polyhydroxy-aldonic acid, (ii) a polyhydroxy-aldonic lactone, (iii) a polyhydroxy-alduronic acid, (iv) a polyhydroxy-alduronic lactone, (v) a polyhydroxy-aldaric acid; (vi) a polyhydroxy-aldaric lactone, and (vii) an organic acid lactone having two or more hydroxyl or chetohydroxyl groups. The dermatological disorder treated is one associated with reactive or dilated blood vessels.

Also included in the invention are methods of treating dermatological disorders associated with reactive or dilated blood vessels that include topical application of a therapeutically effective amount of a cosmetic or dermatological composition. The composition includes at least one compound selected from a compound represented by formula (I):

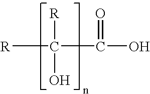

by formula (II):

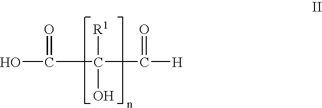

and by formula (III):

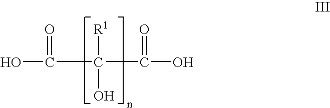

and a lactone derived from an organic acid having two or more hydroxyl or ketohydroxyl groups.

### DETAILED DESCRIPTION OF THE INVENTION

The invention provides a method of treating dermatological disorders that involve or affect the vascular tissues present in the skin, including blood vessels and capillaries. By “disorders involving reactive or dilated blood vessels,” it is meant dermatological disorders that, while not necessarily having as a causative agent a defect or disorder of the blood vessels, involve or relate to an abnormality or dysfunction of the vascular tissues present in the skin, such as the blood vessels or capillaries, including, but not limited to, inflammation, dilation, constriction, or other disturbances of the blood vessels and capillaries of the skin.

Exemplary disorders include, e.g., acanthosis nigricans, acrocyanosis, actinic cheilitis, actinic prurigo, dermatitis, dermatosis, dermographism, dyshidrosis, drug eruptions, eczema, erythema, erythema migrans, erythrocyanosis, erythromelalgia, familial hemorrhage, histamine reaction, inflammatory papular and pustular lesions, lichen planus, lupus erythematosus, mycosis fungoides, neurodermatitis, neuropeptide and neurovascular reactions, parapsoriasis, perniosis (chilblains), photoallergy, photoreaction, photosensitivity, pityriasis rosea, pityriasis rubra pilaris, polymorphic light eruption, psoriasis, rhinophyma, rosacea, sclerosis, spider naevi, T-cell disorders, telangiectasia, urticaria and other vascular reactions involving the blood vessels and/or capillaries of the skin.

The method of the invention includes a topical application of a cosmetic or dermatological composition to an area of skin afflicted with a disorder involving reactive blood vessels. The area of skin may be human skin or animal skin and includes oral and nasal mucosa and nail beds. The compositions applied in the method include at least one of a polyhydroxy-lactone or the free acid, salt, amide, or esters of the same. More than one compound may be in the composition. Preferred are polyhydroxy-lactones or polyhydroxy acids. Any polyhydroxy-lactone known or to be developed in the art may be used in the practice of the method of the invention. Preferably, the selected polyhydroxy-lactone or the free acid, salt, amide, or esters of the same exhibits an anti-oxidation activity as measured by conventional techniques.

As is well understood in the art, the specific polyhydroxy-lactone(s) or the free acid, salt, amide, or esters of the same selected will vary depending on several factors, including the nature of the specific disorder to be treated, the concentration used, the medical condition of the patient, etc. It is generally preferred that the selected component is at least one of:

(a) a polyhydroxy-aldonic acids,

(b) a polyhydroxy-aldonic lactone,

(c) a polyhydroxy-alduronic acids,

(d) a polyhydroxy-alduronic lactone,

(e) a polyhydroxy-aldaric acid,

(f) a polyhydroxy-aldaric lactones, and

(g) a lactone derived from an organic acid which has two or more hydroxyl or ketohydroxyl groups.

For example, the method of the invention may include a polyhydroxy-aldonic acid represented by the generic formula (I):

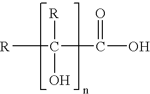

In formula (I), “n” represents an integer of 2 to 30. Preferably “n” is an integer of 2 to 20, more preferably, an integer of 2 to 10, and most preferably an integer of 2 to 6. “R” may be a hydrogen atom, a halogen atom, or any organic hydrocarbon radical, straight chain, branched, or cyclic, substituted or unsubstituted. For example, “R” may be an alkoxy group, an alkyl group, an aralkyl group, or an aryl group. If “R” is an organic hydrocarbon radical, it preferably contains one to fifteen carbon atoms or one to nine carbons atoms.

The selected polyhydroxy-aldonic acid(s) can be present in any stereoisomeric form or in racemic mixtures. For example, the D, L, DL isomers and/or racemic mixtures of the same may be used. The selected polyhydroxy-aldonic acid(s) can be present as saturated or unsaturated, straight or branched chain or cyclic form(s), amide, ester, lactone, or salt form.

Specific polyhydroxy-aldonic acids for use in the method of the invention include, but are not limited to, glyceric acid, erythronic acid, threonic acid, ribonic acid, arabinoic acid, xylonic acid, lyxonic acid, allonic acid, altronic acid, gluconic acid, mannoic acid, gulonic acid, idonic acid, galactonic acid, talonic acid, glucoheptonic acid, galactoheptonic acid, and mannoheptonic acid.

Specific polyhydroxy-aldonic lactones for use in the method of the invention include, but are not limited to erythronolactone, threonolactone, ribonolactone, arabinolactone, xylonolactone, lyxonolactone, allonolactone, altronolactone, gluconolactone, mannolactone, gulonolactone, idonolactone, galactonolactone, talonolactone, glucoheptonolactone, galactoheptonolactone, and mannoheptonolactone.

The method of the invention may include a composition that includes a polyhydroxy-alduronic acid represented by the formula (II):

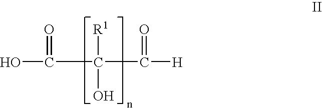

In formula (II), “n” represents an integer of 2 to 30. Preferably “n” is an integer of 2 to 20, more preferably, an integer of 2 to 10, and most preferably an integer of 2 to 6. The group represented by “R¹” may be a hydrogen atom or it may independently be a halogen atom (such as iodine, fluorine, chlorine, or bromine atoms), or an organic hydrocarbon radical, straight chain, branched, or cyclic, substituted or unsubstituted. For example, “Rl” may be an alkoxy group, and alkyl group, an aralkyl group, or an aryl group. If “R¹” is an organic hydrocarbon radical, it preferably contains one to fifteen carbon atoms or one to nine carbons atoms.

The selected polyhydroxy-alduronic acid(s) may be present in any stereoisomeric form or in racemic mixtures. For example, the D, L, DL isomers may be used. The selected polyhydroxy-alduronic acid(s) can be present as saturated or unsaturated, straight or branched chain or cyclic form(s), free acid, amide, ester, lactone, or salt form with organic or inorganic alkali.

Suitable polyhydroxy-alduronic acids may include, without limitation, glyceruronic acid, erythruronic acid, threuronic acid, riburonic acid, arabinuronic acid, xyluronic acid, lyxuronic acid, alluronic acid, altruronic acid, glucuronic acid, mannuroic acid, guluronic acid, iduronic acid, galacturonic acid, taluronic acid, glucohepturonic acid, galactohepturonic acid, and mannohepturonic acid.

Suitable polyhydroxy-alduronic lactones for use in the composition may include, without limitation, erythruronolactone, riburonolactone, arabinuronolactone, xyluronolactone, glucuronolactone, mannuronolactone, guluronolactone, iduronolactone, galacturonolactone, glucohepturonolactone, galactohepturonolactone, and mannohepturonolactone.

The method of the invention includes a composition that incorporates a polyhydroxyaldaric acid represented by the formula (III):

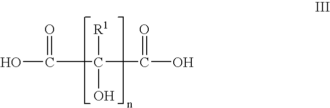

In formula (III), “n” represents an integer of 2 to 30. Preferably “n” is an integer of 2 to 20, more preferably, an integer of 2 to 10, and most preferably an integer of 2 to 6. The group represented by “R¹” may be a hydrogen atom. However, it may also be independently a halogen atom (such as iodine, fluorine, chlorine, or bromine atoms), or an organic hydrocarbon radical, straight chain, branched, or cyclic, substituted or unsubstituted. For example, “R¹” may be an alkoxy group, and alkyl group, an aralkyl group, or an aryl group. If “R¹” is an organic hydrocarbon radical, it preferably contains one to fifteen carbon atoms or one to nine carbons atoms.

Exemplary polyhydroxy-aldaric acids include glyceraric acid, erythraric acid, threaric acid, ribaric acid, arabinaric acid, xylaric acid, lyxaric acid, allaric acid, altraric acid, glucaric acid (saccharic acid), mannaric acid, gularic acid, idaric acid, galactaric acid (mucic acid), talaric acid, glucoheptaric acid, galactoheptaric acid, and mannoheptaric acid.

Suitable polyhydroxy-aldaric lactones include ribarolactone, arabinarolactone, xylarolactone, glucarolactone (saccharolactone), mannarolactone, gularolactone, idarolactone, galactarolactone, glucoheptarolactone, galactoheptarolactone, mannoheptarolactone.

The polyhydroxy-aldaric acid(s) may be present in any stereoisomeric form or in racemic mixtures. For example, the D, L, DL isomers may be used. The selected polyhydroxy-aldaric acid(s) can be present as saturated or unsaturated, straight or branched chain or cyclic form(s), free acid, amide, ester, lactone, salt or partial salt form with organic or inorganic alkali. Examples that can be used in the composition of the method include glucaric diamide, diethyl glucarate, disodium glucate, diammonium glucate, glucaric monoamide, glucaric monoethyl ester, sodium glucate, ammonium glucate, galactaric diamide, diethyl galactate, disodium galactate, galactaric monoamide, galactaric monoethyl ester, sodium galactate and ammonium galactate.

Also useful for inclusion in the composition used in the method of the invention are polyhydroxy-lactones that are derived from organic acids having two or more hydroxyl or ketohydroxyl groups or the organic acid itself, such as ketopolyhydroxy-lactones and acids. Some examples of these compounds include the acids aleuritic acid, glucosaminic acid, galactosaminic acid, mannosaminic acid, hexulosonolactone, 2-keto-gulonolactone, mevalonolactone, pantoic acid, and piscidic acid and the lactones: aleuritic lactone, glucosaminolactone, galactosaminolactone, mannosaminolactone, hexulosonolactone, 2-keto-gulonolactone, mevalonolactone, pantolactone, and piscidolactone.

The composition containing the at least one polyhydroxy-aldonic acid, a polyhydroxy-aldonic lactone, a polyhydroxy-alduronic acid, a polyhydroxy-alduronic lactone, a polyhydroxy-aldaric acid, polyhydroxy-aldaric lactone, and an organic acid lactone having two or more hydroxyl or ketohydroxyl groups may be formulated into any desirable dosage form, so long as such form can be administered topically to the affected area. Suitable formulations are well known in the art, and include suspensions, emulsions (oil-in-water and water-in-oil), pastes, sticks, creams, lotions, gels, solutions, atomizable forms, oils, polymerizing gel formulation, ointments, roll-on sticks, bars, shampoo, spray, powder, masque, and mouth rinse or wash.

As will be understood by a person of skill in the art, the amount of selected polyhydroxy lactone or polyhydroxy acid present in the composition of the invention shall vary, depending on several factors, including, for example, the duration of the prescribed treatment regimen, the specific polyhydroxy lactone(s) or polyhydroxy acid(s) selected for inclusion in the composition, the other ingredients in the composition, the specific nature of the disorder to be treated, including the specific area of skin affected, the dosage form selected and the age, gender, lifestyle, and medical history of the patient. However, in general, the composition of the invention may contain about 0.1% to about 99% by weight of the selected polyhydroxy lactone(s) or polyhydroxy acid(s), with concentrations of about 1% to about 50% by weight, about 2% to about 30% by weight, and about 3% to about 20% by weight being preferred.

In general, because the formulation is to be applied to the skin, it is preferred to have a pH of about three to eight, to minimize irritation, and/or may be in a controlled release formulation, as discussed below. In some cases, an adjustment of the pH of the composition may be desirable because of the tendency of some polyhydroxy-lactones to react with water molecules and form free acid molecules, thereby altering the pH of the overall composition over time until the equilibrium of the reaction has been reached. This lowered pH may cause discomfort to the patient, as the compositions may irritate or sting the skin area when applied. The reaction with water is evidenced by a continued decrease in pH of the solution. As is know in the art, a freshly prepared gluconolactone 1% aqueous solution has pH 3.6. The pH changes to 2.5 after 2 hours. See, The Merck Index, 13^{th} Edition, #4470. Similar results are seen in a 1.78% (0.1 M) aqueous solution of D-gluconolactone, as shown in Table 1:

TABLE 1
pH values over time of a 0.1 M solution of D-gluconolactone.

| Time (in minutes) | pH |
|---|---|
| 0 | 2.1 |
| 60 | 2.0 |
| 360 | 1.9 |
| 4320 | 1.8 |

Most lactones in aqueous solution will behave similarly, as is shown in Table 2:

TABLE 2
Change in pH of Certain Polyhydroxy-Lactones
in Aqueous Solution Over Time

| | Concentration % | Hours | | | | | |
|---|---|---|---|---|---|---|---|
| Polyhydroxy-Lactone | (0.1 M) | Fresh | 1 | 6 | 24 | 48 | 72 |
| D-erythronolactone | 1.18 | 6.3 | 6.3 | 6.2 | 5.5 | 3.2 | 3.1 |
| D-galactonolactone | 1.78 | 5.1 | 5.0 | 3.9 | 3.2 | 2.6 | 2.4 |
| D-gluconolactone | 1.78 | 2.1 | 2.0 | 1.9 | 1.9 | 1.9 | 1.8 |
| D-glucoheptonolactone | 2.08 | 5.6 | 5.6 | 3.9 | 3.1 | 2.5 | 2.3 |
| D-glucuronolactone | 1.76 | 5.7 | 5.7 | 4.8 | 3.4 | 2.6 | 2.3 |
| D-gulonolactone | 1.78 | 6.2 | 5.8 | 4.6 | 3.5 | 3.0 | 2.7 |
| D-pantolactone | 1.30 | 6.5 | 6.5 | 6.4 | 5.9 | 5.7 | 5.7 |
| D-ribonolactone | 1.48 | 6.2 | 6.1 | 5.9 | 3.9 | 2.8 | 2.6 |
| Control | 1.76 | 2.1 | 2.1 | 2.1 | 2.1 | 2.1 | 2.1 |

In Table 2 the control compound is Vitamin C (ascorbic acid).

The pH of compositions of the invention can be raised or lowered by conventional formulation techniques as is known or to be developed in the art. For example, to raise or adjust the pH of a composition containing a polyhydroxy-lactone(s), an organic or inorganic alkali may be used. However, in some cases the bioavailability of the polyhydroxy-lactone or polyhydroxy-acid may be reduced by use of an inorganic alkali for pH adjustment.

Alternatively, the stinging or irritation sensation of a given composition upon contact with the skin may be controlled by reducing the rate at which the acidic compound(s) penetrates the skin. Therefore, in such cases a composition containing a controlled release component may be preferred. Any controlled release component that serves to reduce the rate at which the polyhydroxy-acid penetrates the skin may be used, for example, an amphoteric system or a molecular complex. Amphoteric systems containing amino acids may be used, and are preferred in compositions that contain at least one polyhydroxy-lactone. Preferred amino acids include for use in the amphoteric system include arginine, lysine and ornithine. In addition, the controlled release of the polyhydroxy acid(s) or lactone(s) into the skin can be effected by use of a molecular complex present in the composition. Polar or basic amino acids can be included in the composition to form these molecular complexes. Preferred amino acids are arginine, lysine, histidine tryptophan, and ornithine to form molecular complexes with polyhydroxy-lactones or polyhydroxy-acids.

Other ingredients may be incorporated into these compositions, as is known in the art, to alter the “skin feel,” spreading properties reactive index, or other properties of the formulation. Such ingredients may include colorants, fragrances, emollients, preservatives, etc. For example, the compositions may contain agents added to enhance the cosmetic, pharmaceutical, or other properties of the composition. Any such agent(s) may be included as long as they are incorporated in such as manner that the therapeutic properties of the polyhydroxy acids or polyhydroxy lactones are not impeded. In most cases, inclusion of more than one agent is desirable.

Agents may include an agent that improves or eradicates age spots, keratoses and wrinkles; a local analgesic; a local anesthetic; an antiacne agent; an antibacterial agent; an antiyeast agent; an antifungal agent; an antiviral agent; an antidandruff agent; an antidermatitis agent; an antihistamine; an antipruritic agent; an antiemetic; an antimotionsickness agent; an antiinflammatory agent; an antihyperkeratotic agent; an antiperspirant; an antipsoriatic agent; an antiseborrheic agent; a conditioner; an antiaging agent; an antiwrinkle agent; a sunblock; a sunscreen; a skin lightening agent; a depigmenting agent; a vitamin; a corticosteroid; a tanning agent; a humectant; a hormone; a retinoid; a gum disease or oral care agent; a topical cardiovascular agent; a corn, callus and/or wart removing agent; a depilating agent; and/or other dermatologicals.

Additional exemplary agents include aclovate, acyclovir, acetylsalicylic acid, adapalene, albuterol, aluminum acetate, aluminum chloride, aluminum hydroxide, aluminum chlorohydroxide, amantadine, aminacrine, aminobenzoic acid (PABA), aminocaproic acid, aminosalicylic acid, amitriptyline, anthralin, ascorbic acid, ascorbyl palmitate, atropine, azelaic acid, bacitracin, bemegride, beclomethasone dipropionate, benzophenone, benzoyl peroxide, betamethasone dipropionate, betamethasone valerate, brompheniramine, bupivacaine, butoconazole, calcipotriene, camphor, capsaicin, carbamide peroxide, chitosan, chlorhexidine, chloroxylenol, chlorpheniramine, ciclopirox, clemastine, clindamycin, clioquinol, clobetasol propionate, clotrimazole, coal tar, cromolyn, crotamiton, cycloserine, dehydroepiandrosterone, desoximetasone, dexamethasone, diphenhydramine, doxypin, doxylamine, dyclonine, econazole, erythromycin, estradiol, ethinyl estradiol, fluocinonide, fluocinolone acetonide, 5-fluorouracil, griseofulvin, guaifenesin, haloprogin, hexylresorcinol, homosalate, hydrocortisone, hydrocortisone 21-acetate, hydrocortisone 17-valerate, hydrocortisone 17-butyrate, hydrogen peroxide, hydroquinone, hydroquinone monoether, hydroxyzine, ibuprofen, ichthammol, imiquimod, indomethacin, ketoconazole, ketoprofen, kojic acid, lidocaine, meclizine, meclocycline, menthol, mepivacaine, methyl nicotinate, methyl salicylate, metronidazole, miconazole, minocycline, minoxidil, monobenzone, mupirocin, naftifine, naproxen, neomycin, nystatin, octyl methoxycinnamate, octyl salicylate, oxybenzone, oxiconazole, oxymetazoline, padimate O, permethrin, pheniramine, phenol, phenylephrine, phenylpropanolamine, piperonyl butoxide, podophyllin, podofilox, povidone iodine, pramoxine, prilocaine, procaine, promethazine propionate, propranolol, pseudoephedrine, pyrethrin, pyrilamine, resorcinol, retinal, 13-cis retinoic acid, retinoic acid, retinol, retinyl acetate, retinyl palmitate, salicylamide, salicylic acid, selenium sulfide, shale tar, sulconazole, sulfuir, sulfadiazine, tazarotene, terbinafine, terconazole, tetracaine, tetracycline, tetrahydrozoline, thymol, tioconazole, tolnaftate, triamcinolone diacetate, triamcinolone acetonide, triamcinolone hexacetonide, triclosan, triprolidine, undecylenic acid, urea, vitamin E acetate, wood tar, and zinc pyrithione.

Moreover, other types of hydroxyacids, ketoacids, and related compounds may be includes in the composition, such as hydroxymonocarboxylic acids, hydroxydicarboxylic acids, 2-hydroxycarboxylic acids, hydroxycarboxylic acids, 2-ketocarboxylic acids and compounds listed in U.S. Pat. Nos. 5,422,370, 5,547,988, 5,470,880, and 5,385,938, the contents of each of which are incorporated herein by reference.

Phenyl alpha acyloxyalkanoic acids and derivatives thereof may be incorporated into the composition, including, but not limited to diphenyl alpha acetoxyacetic acid, phenyl alpha acetoxyacetic acid, phenyl alpha methyl alpha acetoxyacetic acid, phenyl alpha acetoxypropanoic acid, and 2-phenyl beta acetoxypropanoic acid. These compounds may exist in a free acid, or salt form, or as stereoisomers. Such compounds are listed, e.g., in U.S. Pat. Nos. 5,258,391 and 5,643,949, the contents of each of which are incorporated herein by reference.

N-acetyl aldosamines and N-acetylamino acids as described in, e.g., U.S. Pat. Nos. 6,159,485 and 6,524,593 (the contents of each of which are incorporated herein by reference) may be incorporated in the compositions. N-acetyl-cysteine, N-acetyl-proline, N-acetyl-glutamine and N-acetyl-glucosamine may be preferred. These compounds may exist in a free acid, or salt form, or as a stereoisomer or non-stereoisomer.

### General Preparation of Compositions for Use in the Methods of the Invention

In each formulation described below, the amount of polyhydroxy-lactone(s) and/or acid(s) used will vary depending on the final concentration desired, within the parameters discussed above.

To prepare the composition in the form of a solution, the selected polyhydroxy-lactone(s) is dissolved in any solvent that is acceptable for topical administration. For example, one may use water, ethanol, propylene glycol, butylene glycol, or mixtures of these. To prepare a topical composition in lotion, cream or ointment form, the selected polyhydroxy-lactone(s) and/or acid(s) is first dissolved in any solvent that is acceptable for topical administration (water, ethanol, propylene glycol being preferred). This solution is mixed with a desired base or additional pharmaceutically acceptable vehicle to make lotion, cream or ointment.

A topical composition of the instant invention may also be formulated in a gel or shampoo form. A typical gel composition is formulated by the addition of a gelling agent such as chitosan, methyl cellulose, ethyl cellulose, polyvinyl alcohol, polyquaterniums, hydroxyethylcellulose, hydroxypropylcellulose, hydroxypropylmethylcellulose, carbomer or ammoniated glycyrrhizinate to a solution comprising the polyhydroxy-lactone(s). The preferred concentration of the gelling agent may range from 0.1 to 4 percent by weight of the total composition.

In the preparation of shampoo, a polyhydroxy-lactone is first dissolved in water or propylene glycol, and the solution is mixed with a shampoo base.

Other forms of compositions for delivery of a polyhydroxy-lactone of the instant invention are readily blended, prepared or formulated by those skilled in the art.

### PREPARATION EXAMPLE 1

### Cream and Ointment Form of Composition

A cream form composition and an ointment form composition containing D-gluconolactone were prepared. D-gluconolactone (24 g) was dissolved in a solution of water (36 ml) and propylene glycol (10 ml). The solution was mixed and divided into two equal aliquots. The first aliquot was mixed with a cream base (65 g). The second was mixed with a hydrophilic ointment (65 g). Each of the finished cream and ointment had a pH of 1.8 and contained 12% D-gluconolactone by weight.

### PREPARATION EXAMPLE 2

### Emulsion Form of Composition

An emulsion form composition containing D-gluconolactone was prepared. A commercial oil-in-water emulsion cream base was obtained. A first solution containing 10 g D-gluconolactone dissolved in water (20 ml) and propylene glycol (10 ml) was prepared. A second solution was prepared in the same way, except that D-gluconolactone was addend in an amount of 20 g. Each of the first and the second solutions was individually mixed with 60 g or 50 g, respectively, of the oil-in-water emulsion cream base. The two resulting emulsions contained 10% by weight and 20% by weight of D-gluconolactone.

### PREPARATION EXAMPLE 3

### Solution Form of Composition

D-gluconic acid 50% aqueous solution 36 g was mixed with alcohol (20 ml). The solution had a pH of 1.7 and contained 36% by weight of D-gluconic acid.

### PREPARATION EXAMPLE 4

### Solution Form of Composition

D-gluconolactone (30 g), N-acetyl-L-proline (10 g), and N-acetyl-D-glucosamine (10 g) were dissolved in water (80 ml), propylene glycol (40 ml), alcohol (20 ml), and oleyl lactate (10 ml). The finished solution had a pH of 1.8 and contained 20% D-gluconolactone by weight.

### PREPARATION EXAMPLE 5

### Cream and Ointment Form Preparation

D-Glucuronolactone (20 g) was dissolved in water (50 ml) and propylene glycol (10 ml). This solution was divided into two equal aliquots. The first aliquot was mixed with a cream base (60 g). The second aliquot was mixed with a hydrophilic ointment (60 g). The resulting ointment had a pH of 4.0 and contained 10% glucuronolactone.

### PREPARATION EXAMPLE 6

### Lotion Form of Composition

D-glucuronolactone 15% oil-in-water lotion was prepared. D-glucuronolactone (15 g) was dissolved in water (30 ml) and propylene glycol (15 ml). The resulting solution was mixed with oil-in-water lotion base (40 g).

### PREPARATION EXAMPLE 7

### Cream Form Preparation

D-galacturonic acid (10 g) was dissolved in water (20 ml) and propylene glycol (6 ml). The solution was mixed with a cream base (64 g). The finished cream had a pH of 1.7 and contained 10% D-galacturonic acid.

### PREPARATION EXAMPLE 8

### Cream Form of Composition

D-erythronolactone (2.4 g) was dissolved in water (4 ml) and propylene glycol (2.4 ml). The solution was mixed with a cream base (21.2 g). The finished cream had a pH of 3.0 and contained 8% D-erythronolactone.

### PREPARATION EXAMPLE 9

### Ointment Form of Composition

D-ribonolactone (10 g) was dissolved in water (20 ml) and propylene glycol (6 ml). The solution was mixed with a hydrophilic ointment (64 g). The finished ointment had a pH of 2.9 and contained 10% D-ribonolactone.

### PREPARATION EXAMPLE 10

### Ointment Form of Composition

D-galactonolactone (10 g) was dissolved in water (20 ml) and propylene glycol (6 ml). The solution was mixed with a cream base or hydrophilic ointment (64 g). The ointment had a pH of 3.1 and contained 10% D-galactonolactone.

### PREPARATION EXAMPLE 11

### Solution Form of Composition

D-glucoheptonolactone (2 g) was dissolved in water (98 ml). The finished solution had a pH of 5.6 and contained 2% D-glucoheptonolactone.

### PREPARATION EXAMPLE 12

### Solution Form of Composition

D-pantolactone (1 g) was dissolved in water (99 ml). The solution thus obtained had a pH of 6.5 and contained 1% D-pantolactone.

### PREPARATION EXAMPLE 13

### Emulsion Form of Composition

DL-glyceric acid (40%) aqueous solution (25 g) was mixed with oil-in-water emulsion (75 g). The finished emulsion had a pH pf 1.8 and contained 10% DL-glyceric acid.

### PREPARATION EXAMPLE 14

### Cream Form of Composition

D-glucuronoamide (10 g) was dissolved in warm water (30 ml), and the solution was mixed with an oil-in-water emulsion (60 g). The cream had a pH of 3.1 and contained 10% D-glucuronoamide.

### USE EXAMPLE 1

A female patient, age 57, having distended blood capillary vessels on the face and nose for several years duration, topically applied twice daily a D-gluconolactone 10% oil-in-water cream. At the end of five weeks, the appearance of the capillary vessels improved significantly that the patient's facial skin and skin of nose appeared normal, thus demonstrating that the method is therapeutically effective for topical treatment of distended blood vessels.

### USE EXAMPLE 2

A male patient, age 84, with chronic plaque psoriasis developed persistent facial redness due to distended blood capillary vessels. The subject topically applied twice daily a D-gluconolactone 15% oil-in-water lotion on his face for one month. At the end of one month, the appearance of the capillary vessels improved significantly that the patient's facial skin appeared normal. This result shows that the method is therapeutically effective for topical treatment of distended blood vessels.

### USE EXAMPLE 3

A male patient, age 87, with history of occupational exposure to sunlight developed large dilated blood vessels on his nose, sides of face and temporal areas. The patient topically applied twice daily the D-gluconolactone 16% oil-in-water lotion on his face and his nose for two and half months. At the end of two and half months, the dilated capillaries and venules appeared to be substantially improved such that the patient's face appeared only slightly reddish. This result shows that the method is therapeutically effective for topical treatment of dilated blood vessels.

### USE EXAMPLE 4

A female patient, age 77, with an apparent genetic condition has redness of cheeks, nose and chin, topically applied twice daily the D-glucuronolactone 15% oil-in-water lotion on her face and her nose for three months. At the end of three months, the redness of her face diminished significantly and the clinical evaluation was judged to be a 90% improvement. This result shows that the method is therapeutically effective for topical treatment of dilated blood vessels.

### USE EXAMPLE 5

A female patient, age 26, having severe actinic erythema of lips with dryness and tenderness for three days due to sunlight, cold air and wind during skiing, topically applied several times daily the D-gluconolactone 10% oil-in-water cream on her lips for three days. At the end of three days, the actinic erythema disappeared completely and the clinical evaluation was judged to be 100% improvement. This result shows that the method is therapeutically effective for topical treatment of actinic erythema.

### USE EXAMPLE 6

A male patient, age 51, with chronic seborrheic eczema for seven years had symptoms and signs including pruritus, scaliness and intense erythema of his entire face. Topical applications of corticosteroids only partially relieved his symptoms and signs. The subject topically applied twice daily the D-gluconolactone 20% oil-in-water formulation on his face. The symptoms and signs improved substantially, and his conditions have been controlled over the following two years with the above formulation. This result shows that the method is therapeutically effective for topical treatment of seborrheic eczema.

### USE EXAMPLE 7

A male patient, age 71, having nummular eczema on his left forearm, topically applied twice daily the above D-gluconolactone 12% cream on itchy erythematous lesions. The itch disappeared quickly, the erythema gradually diminished, and the lesions improved substantially over the next week. This result shows that the method is therapeutically effective for topical treatment of eczema.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A method of topically treating a dermatological disorder comprising topically applying a therapeutically effective amount of a cosmetic or dermatological composition to an affected area of the skin, wherein the composition comprises at least one compound selected from a polyhydroxy-aldonic acid, a polyhydroxy-aldonic lactone, a polyhydroxy-alduronic acid, a polyhydroxy-alduronic lactone, a polyhydroxxy-aldaric acid, polyhydroxy-aldaric lactone, and an organic acid lactone having two or more hydroxyl or ketohydroxyl groups; and the dermatological disorder is one associated with reactive or dilated blood vessels.

2. The method of claim 1, wherein the composition further comprises a topical agent.

3. The method of claim 1, wherein the dermatological disorder is selected from the group consisting of acrocyanosis, erythema, erythrocyanosis, erythromelalgia, familial hemorrhage, perniosis (chilblains), spider naevi, telangiectasia, and vascular reactions.

4. The method of claim 1, wherein the dermatological disorder is selected from dermatitis, dermatosis, dermographism, eczema, histamine reaction, lupus erythematosus, mycosis fungoides, parapsoriasis, rhinophyma, rosacea, T-cell disorders, psoriasis, lichen sclerosis.

5. The method of claim 1, wherein the dermatological disorder is selected from the group consisting of actinic cheilitis, actinic prurigo, drug eruptions, erythema migrans, photoallergy, photoreaction, photosensitivity,

6. The method of claim 1, wherein the dermatological disorder is selected from the group consisting of acanthosis nigricans, inflammatory papular and pustular lesions, dyshidrosis, lichen planus, neurodermatitis, neuropeptide and neurovascular reactions, pityriasis rosea, pityriasis rubra pilaris, polymorphic light eruption, and urticaria.

7. The method of claim 1, wherein the at least one compound is selected from the group consisting of glyceric acid, erythronic acid, threonic acid, ribonic acid, arabinoic acid, xylonic acid, lyxonic acid, allonic acid, and altronic acid.

8. The method of claim 1, wherein the at least one compound is selected from the group consisting of gluconic acid, mannoic acid, gulonic acid, idonic acid, galactonic acid, talonic acid, glucoheptonic acid, galactoheptonic acid, and mannoheptonic acid.

9. The method of claim 1, the at least one compound is selected from the group consisting of erythronolactone, threonolactone, ribonolactone, arabinolactone, xylonolactone, lyxonolactone, allonolactone, altronolactone, and gluconolactone.

10. The method of claim 1, wherein the at least one compound is selected from the group consisting of mannolactone, gulonolactone idonolactone, galactonolactone, talonolactone, glucoheptonolactone, galactoheptonolactone, and mannoheptonolactone.

11. The method of claim 1, wherein the at least one compound is selected from the group consisting of glyceruronic acid, erythruronic acid, threuronic acid, riburonic acid, arabinuronic acid, xyluronic acid, lyxuronic acid, alluronic acid, and altruronic acid.

12. The method of claim 1, wherein the at least one compound is selected from the group consisting of glucuronic acid, mannuroic acid, guluronic acid, iduronic acid, galacturonic acid, taluronic acid, glucohepturonic acid, galactohepturonic acid, and mannohepturonic acid.

13. The method of claim 1, wherein the at least one compound is selected from the group consisting of erythruronolactone, riburonolactone, arabinuronolactone, xyluronolactone, glucuronolactone, and mannuronolactone.

14. The method of claim 1, wherein the at least one compound is selected from the group consisting of guluronolactone, iduronolactone, galacturonolactone, glucohepturonolactone, galactohepturonolactone, and mannohepturonolactone.

15. The method of claim 1, wherein the at least one compound is selected from the group consisting of glyceraric acid, erythraric acid, threaric acid, ribaric acid, arabinaric acid, xylaric acid, lyxaric acid, allaric acid, and altraric acid.

16. The method of claim 1, wherein the at least one compound glucaric acid (saccharic acid), mannaric acid, gularic acid, idaric acid, galactaric acid (mucic acid), talaric acid, glucoheptaric acid, galactoheptaric acid, and mannoheptaric acid.

17. The method of claim 1, wherein the at least one compound is selected from glucaric diamide, diethyl glucarate, disodium glucate, diammonium glucate, glucaric monoamide, glucaric monoethyl ester, sodium glucate, and ammonium glucate.

18. The method of claim 1, wherein the at least one compound is selected from galactaric diamide, diethyl galactate, disodium galactate, galactaric monoamide, galactaric monoethyl ester, sodium galactate, and ammonium galactate.

19. The method of claim 1, wherein the at least one compound is selected from ribarolactone, arabinarolactone, xylarolactone, glucarolactone (saccharolactone), mannarolactone, gularolactone, idarolactone, galactarolactone, glucoheptarolactone, galactoheptarolactone, mannoheptarolactone.

20. The method of claim 1, wherein the at least one compound is selected from the group consisting of aleuritic acid, glucosaminic acid, galactosaminic acid, mannosaminic acid, hexulosonic acid, 2-keto-gulonic acid, mevalonic acid, pantoic acid, and piscidic acid.

21. The method of claim 1, wherein the at least one compound is selected from the group consisting of aleuritic lactone, glucosaminolactone, galactosaminolactone, mannosaminolactone, hexulosonolactone, 2-keto-gulonolactone, mevalonolactone, pantolactone, and piscidolactone.

22. The method of claim 2, wherein the agent is selected from the group consisting of an agent that improves or eradicates age spots, keratoses and wrinkles, a local analgesic, a local anesthetic; an antiacne agent; an antibacterial agent; an antiyeast agent; and antifungal agents; and antiviral agent; an antidandruff agents; an antidermatitis agent; an antihistamine; an antipruritic agent; and an antiemetic.

23. The method of claim 2, wherein the agent is selected from the group consisting of an antimotionsickness agent; an antiinflammatory agent; an antihyperkeratotic agent; an antiperspirant; an antipsoriatic agent; an antiseborrheic agent; a conditioner, an antiaging agent, an antiwrinkle agent; a sunblock agent; a sunscreen agent; a skin lightening agent; and a depigmenting agent.

24. The method of claim 2, wherein the agent is selected from the group consisting of a vitamin; a corticosteroid; a tanning agent; a humectant; a hormone; a retinoid; a gum disease or oral care agent; a topical cardiovascular agent; a corn, callus and wart removing agent; and a dipilating agent.

25. The method of claim 2, wherein the agent is selected from the group consisting of aclovate, acyclovir, acetylsalicylic acid, adapalene, albuterol, aluminum acetate, aluminum chloride, aluminum hydroxide, aluminum chlorohydroxide, amantadine, aminacrine, aminobenzoic acid (PABA), aminocaproic acid, aminosalicylic acid, amitriptyline, anthralin, ascorbic acid, ascorbyl palmitate, atropine, azelaic acid, bacitracin, bemegride, beclomethasone dipropionate, benzophenone, benzoyl peroxide, betamethasone dipropionate, betamethasone valerate, and brompheniramine.

26. The method of claim 2, wherein the agent is selected from the group consisting of bupivacaine, butoconazole, calcipotriene, camphor, capsaicin, carbamide peroxide, chitosan, chlorhexidine, chloroxylenol, chlorpheniramine, ciclopirox, clemastine, clindamycin, clioquinol, clobetasol propionate, clotrimazole, coal tar, cromolyn, crotamiton, cycloserine, dehydroepiandrosterone, desoximetasone, dexamethasone, diphenhydramine, doxypin, doxylamine, dyclonine, econazole, erythromycin, estradiol, ethinyl estradiol, fluocinonide, fluocinolone acetonide, 5-fluorouracil, griseofulvin, guaifenesin, haloprogin, hexylresorcinol, homosalate, hydrocortisone, hydrocortisone 21-acetate, hydrocortisone 17-valerate, hydrocortisone 17-butyrate, hydrogen peroxide, hydroquinone, hydroquinone monoether, hydroxyzine, ibuprofen, ichthammol, imiquimod, indomethacin, ketoconazole, ketoprofen, kojic acid, lidocaine, meclizine, meclocycline, menthol, mepivacaine, methyl nicotinate, methyl salicylate, metronidazole, miconazole, minocycline, minoxidil, monobenzone, mupirocin, naftifine, naproxen, neomycin, nystatin, octyl methoxycinnamate, octyl salicylate, oxybenzone, oxiconazole, oxymetazoline, padimate O, permethrin, pheniramine, phenol, phenylephrine, phenylpropanolamine, piperonyl butoxide, podophyllin, podofilox, povidone iodine, pramoxine, prilocaine, procaine, promethazine propionate, propranolol, pseudoephedrine, pyrethrin, pyrilamine, resorcinol, retinal, 13-cis retinoic acid, retinoic acid, retinol, retinyl acetate, retinyl palmitate, salicylamide, salicylic acid, selenium sulfide, shale tar, sulconazole, sulfur, sulfadiazine, tazarotene, terbinafine, terconazole, tetracaine, tetracycline, tetrahydrozoline, thymol, tioconazole, tolnaftate, triamcinolone diacetate, triamcinolone acetonide, triamcinolone hexacetonide, triclosan, triprolidine, undecylenic acid, urea, vitamin E acetate, wood tar, and zinc pyrithione.

27. The method of claim 2, wherein the agent is selected from the group consisting of a hydroxyacid, a ketoacid, hydroxymonocarboxylic acid, hydroxydicarboxylic acid, 2-hydroxycarboxylic acid, 2-ketocarboxylic acid, a phenyl alpha acyloxyalkanoic acid and derivatives thereof, diphenyl alpha acetoxyacetic acid, phenyl alpha acetoxyacetic acid, phenyl alpha methyl alpha acetoxyacetic acid, phenyl alpha acetoxypropanoic acid, 2-phenyl beta acetoxypropanoic acid, an N-acetyl aldosamine, an N-acetylamino acid, N-acetyl-cysteine, N-acetyl-proline, N-acetyl-glutamine, and N-acetyl-glucosamine.

28. A method of topically treating a dermatological disorder comprising topically applying a therapeutically effective amount of a cosmetic or dermatological composition to an affected area of the skin, wherein the composition comprises at least one compound selected from:
a compound represented by formula (I): 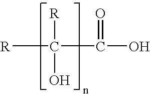
wherein n is an integer of 2 to 30; R is independently one of a hydrogen atom, a halogen atom, an alkoxy group, an alkyl group, an aralkyl group, and an aryl group;
a compound represented by formula (II): 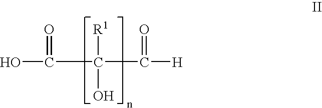
wherein n is an integer of 2 to 30 and R¹ is independently one of a hydrogen atom, a halogen atom, an alkyl group, an alkoxyl group, and an aryl group;
a compound represented by formula (III): 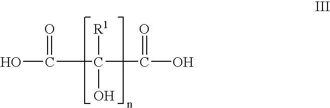
wherein n is an integer of 2 to 30 and R¹ is independently one of a hydrogen atom, a halogen atom, an alkyl group, an alkoxyl group, and an aryl group; and
a lactone derived from an organic lactone having two or more hydroxyl or ketohydroxyl groups;
and the dermatological disorder is one associated with reactive or dilated blood vessels.

29. The method of claim 1, wherein polyhydroxy-aldonic acid is selected from the group consisting of ribonic acid, gluconic acid, galactonic acid and glucoheptonic acid.

30. The method of claim 1, wherein polyhydroxy-aldonic lactone is selected from the group consisting of ribonolactone, gluconolactone, galactonolactone, gulonolactone and glucoheptonolactone.

31. The method of claim 1, wherein polyhydroxy-alduronic acid is selected from the group consisting of glucuronic acid, galacturonic acid and iduronic acid.

32. The method of claim 1, wherein polyhydroxy-alduronic lactone is selected from the group consisting of glucuronolactone, galacturonolactone and iduronolactone.

33. The method of claim 1, wherein polyhydroxy-aldaric acid is selected from the group consisting of glucaric acid, galactaric acid and glucoheptaric acid.

34. The method of claim 1, wherein polyhydroxy-aldaric lactone is selected from the group consisting of glucarolactone, galactarolactone and glucoheptarolactone.
